(19) United States
(12) Patent Application Publication
(10) Pub. No.: US 2002/0128497 A1
(43) Pub. Date: Sep. 12, 2002

(54) Method for the preparation of citalopram

(75) Inventors: Eva Bolzonella, Padova (IT); Andrea Castellin, Mistrino (IT); Andrea Nicole, Padova (IT)

Correspondence Address: DARBY & DARBY P.C., POST OFFICE BOX 5257, NEW YORK, NY, 10150-5257, US

(73) Assignee: H. Lundbeck A/S, Valby-Copenhagen (DK)

(21) Appl. No.: 10/096,149

(22) Filed: Mar. 6, 2002

Related U.S. Application Data
(63) Continuation of application No. PCT/DK01/00481, filed on Jul. 6, 2001.

(30) Foreign Application Priority Data
Jul. 6, 2000 (EP) PCT/EP00/06426

(51) Int. Cl. C07D 307/87

(52) U.S. Cl. 549/467

(57) ABSTRACT

A method for the manufacture of citalopram characterized in (i) reaction of 1-(4-fluorophenyl)-1-(3-dimethylaminopropyl)-5-halophthalane with an activated magnesium to form the Grignard reagent [3-[1-(4-fluorophenyl)-1,3 dihydro-isobenzofuran-1-yl]propyl]dimethylamine 5-magnesium halide followed by (ii) reaction of [3-[1-(4-fluorophenyl)-1,3 dihydro-isobenzofuran-1-yl]propyl)dimethylamine 5-magnesium halide with a compound containing a —CN group bound to a leaving group to form citalopram.

The present invention relates to a method for the preparation of the well-known antidepressant drug citalopram,1-[3-(dimethylamino)propyl]-1-(4-fluorophenyl)-1,3-dihydro-5-isobenzofuran-carbonitrile.

### BACKGROUND OF THE INVENTION

Citalopram is a well-known antidepressant drug that has now been on the market for some years and has the following structure:

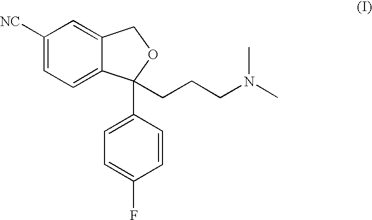

It is a selective, centrally acting serotonin (5-hydroxytryptamine; 5-HT) reuptake inhibitor, accordingly having antidepressant activities. The antidepressant activity of the compound has been reported in several publications, eg. J. Hyttel Prog. Neuro-Psychopharmacol. & Biol. Psychiat. 1982, 6, 277-295 and A. Gravern Acta Psychiatr. Scand. 1987, 75,478-486. The compound has further been disclosed to show effects in the treatment of dementia and cerebrovascular disorders, EP-A-474580.

Citalopram was first disclosed in DE 2,657,013, corresponding to U.S. Pat. No. 4,136,193. This patent publication describes the preparation of citalopram by one method and outlines a further method which may be used for preparing citalopram.

According to the process described, the corresponding 1-(4fluorophenyl)-1,3-dihydro-5-isobenzofurancarbonitrile is reacted with 3-(N,N-dimethylamino)propyl-chloride in the presence of methylsulfinylmethide as condensing agent. The starting material was prepared from the corresponding 5-bromo derivative by reaction with cuprous cyanide.

International patent application No. WO 98/019511 discloses a process for the manufacture of citalopram wherein a (4-(cyano, alkyloxycarbonyl or alkylaminocarbonyl)-2-hydroxymethylphenyl-(4-fluorophenyl)methanol compound is subjected to ring closure. The resulting 5-(alkyloxycarbonyl or alkylaminocarbonyl)-1-(4-fluorophenyl)-1,3-dihydroisobenzofiiran is converted to the corresponding 5-cyano derivative and the 5-cyano derivative is then alkylated with a (3-dimethylamino)propylhalogenide in order to obtain citalopram.

It has now, surprisingly, been found that citalopram may be manufactured by a novel, favourable process characterised by the conversion of 1-(4′-fluorophenyl)-3-(dimethylaminopropyl)-5-halophtalane into the corresponding Grignard reagent; this intermediate is then converted into citalopram by reaction with compounds containing a cyano group bound to a leaving group. The process enables citalopram to be obtained in high yields and does not involve the use of drastic temperature conditions.

### SUMMARY OF THE INVENTION

The object of the present invention is a method for the preparation of citalopram characterised by the following steps:

(i) Reaction of 1-(4′-fluorophenyl)-1-(3-dimethylaminopropyl)-5-halophtalane having the formula

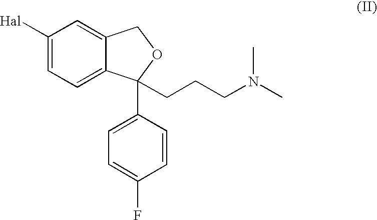

wherein Hal is halogen, i.e chloro, bromo, fluoro and iodo with activated magnesium, to form the Grignard reagent having formula (HI)

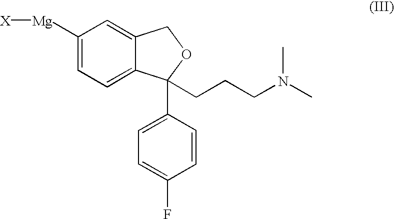

where X is a halogen atom, preferably bromine; and

(ii) Reaction of the intermediate Grignard product (m), with a compound that contains a —CN group bound to a leaving group, to form citalopram.

Step (i)

Step (i) of the process of the invention consist in the conversion of 1-(4′-fluorophenyl)-1-(3-dimethylamionopropyl)-5-halopthalane (II) into the Grignard reagent of formula ([1) by reaction of the compound of formula (II) with activated magnesium. The term “halophtalane” means a derivative of formula (I) in which the “Hal” group is an atom chosen from among bromine, fluorine, chlorine and iodine.

The compound (II) is easily synthesizeable, for example as described in GB-A-1526 331.

The activated magnesium to be used is obtainable by conventional procedures, for example by reaction of metallic magnesium chips with bromoethane or 1,2-dibromoethane, in an ether solvent such as ethyl ether, tetrahydrofuran or 2-methyltetrahydrofuran, possibly in a mixture with toluene or other inert solvents, at a temperature between 25° and the reflux temperature of the mixture.

According to a particular embodiment of the invention, a solution of the compound (II) in an organic solvent, for example in tetrahydrofuran, (hereinafter defined “solution b”) is slowly added to the mixture of a solvent and activated magnesium obtained as described above (hereinafter defined “solution a”). The temperature of the reaction mixture is suitably kept between 40° C. and 65° C.

In order to obtain high yields of the desired product, the following conditions of reaction have been found particularly important:

The compound (II) is used in a molar ratio with respect to magnesium between 3:1 and 1:1, preferably 1:2;

The concentration of compound (II) in “solution b” is between 0.7 M and 1.2 M, preferably 1 M;

The volume of “solution a” is between 40% and 60%, preferably 50%, with respect to the volume of “solution b”;

The time within which “solution b” is added is higher than 5 hours, and is preferably between 6 and 8 hours.

Step (ii)

According to step (ii), the Grignard intermediate of formula (E) is reacted with a compound that contains a —CN group bound to a leaving group, wherein the —CN group acts as an electrophilic group. Examples of such compounds are:

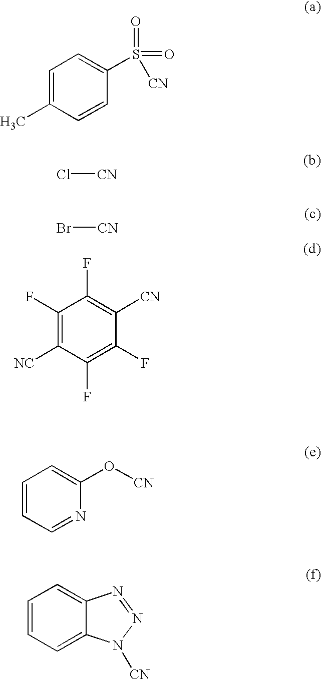

Among these, particularly preferred are the derivatives of formulas (b), (e) and (f). The aforesaid compound that contains a —CN group bound to a leaving group is dissolved in an organic solvent, for example tetrahydrofuran, and is added to a solution of compound (II); preferably the solution of compound (III) has been added a zinc salt, e.g. ZnBr or ZnCl. The compound that contains a —CN group bound to a leaving group is used in a molar ration preferably of 2:1, approximately, with respect to compound (D).

Citalopram (1) is obtained from the reaction mixture through appropriate extractions and washings.

The method according to the invention enables citalopram to be obtained in a high yield and without drastic conditions of temperature. The aforesaid method presents the further advantage of not being racemizing. Consequently, if the starting product of formula (II) is used in an enantiomerically pure form (for example the S-form), it is possible to obtain the corresponding enantiomer of citalopram (i.e. escitalopram) directly, without any need to separate the isomers, hence without any loss of product in the form of undesired enantiomer and with a corresponding increase in yield.

The invention is now illustrated via the following experimental examples, which are provided purely as non-limiting examples.

### EXAMPLE 1

Grignard Reagent of 1-(4′-fluoro)-1-(3-dimethylaminopropyl)5-bromophtalane

In an inert atmosphere and under vigorous stirring, to a suspension of 150 g (6.17 mol) of magnesium chips in 1500 nL of tetrahydrofura are added, at a temperature of 30-35° C., 15 mL (21.9 g, 0.20 mol) of bromoethane. Upon activation of the magnesium, detected by spontaneous exothermia and foaming of the reaction mixture, at the temperature of 55° C. an approximately 1 molar solution of 1125 g (2.98 mol) of 1-(4′-fluorophenyl)-1-(3-dimethylaminopropyl)-5-bromophtalane in 3000 mL of tetrahydroftiran starts to percolate over a period of 7 hours. The reaction mixture is kept spontaneously refluxed throughout the addition. The mixture containing the Grignard reagent thus obtained is used in the subsequent phase of synthesis, after prior cooling at a temperature of approximately 20° C.

### EXAMPLE 2

[3-[1-(4-fluorophenyl)-1,3 dihydro-isobenzofuran-1-ylgpropyl]dimethyl amine 5-magnesium Bromide

The synthesis of the Grignard reagent is carried out as described in example 1 starting from 72 g of [3-[1-(4-fluorophenyl)-1,3 dihydro-isobenzofuran-1-yl]propyl]dimethyl amine (0.19 mol).

The mixture containing the Grignard reagent thus obtained is used in the subsequent phase of synthesis, after prior cooling to 20° C.

### EXAMPLE 3

Synthesis of Citalopram from Tosyl Cyanide

In an inert atmosphere and with perfectly dehydrated apparatus at room temperature, a solution of 2.76 g of p-toluen sulphonyl cyanide (30 mmol) is prepared in 50 mL of anhydrous tetrahydrofuran. The solution thus obtained is brought to the temperature of −20° C. and at this temperature there is added, drop by drop and under vigorous stirring, 50 mL of a solution of [3-[1-(4-fluorophenyl)-1,3 dihydro-isobenzofuran-1-yllpropyl]dimethylamine 5-magnesium bromide (15 mmol) in tetrahydrofuran, obtained as described in example 1, starting from 5.7 g of 13-[1-(4-fluorophenyl)-1,3 dihydro-isobenzofuran-1-yl]propyl]dimethylaniine. Once the addition is completed, the solution obtained is kept at a temperature of −20 IC for 30 minutes and then brought to 20° C. The reaction is then extinguished by percolation of the solution in a mixture of 50 g of 30% anmmonia and ice, subsequently being brought to room temperature to enable decomposition of the non-reacted p-toluenesulphonyl cyanide. The mixture is then neutralized with diluted hydrochloric acid (1 molar) and extracted with 4 aliquots of 75 mL toluene. The reunited organic extracts are washed with 2 aliquots of 100 mL of a saturated solution of sodium chloride, dehydrated with MgSO₄ and concentrated at reduced pressure, to obtain a dark-red oily residue.

The crude mixture obtained is then purified by flash chromatography on 50 g of silica gel 70-230 mesh (eluent: toluene-isopropanol-triethylarnine, 95-5-2, v/v) to obtain 2.54 g of pure product (molar yield 52.3%) having an NMR profile in accordance with the desired structure.

### EXAMPLE 4

Synthesis of Citalopram from 1-cyanobenzotriazol

In an inert atmosphere and with perfectly dehydrated apparatus at room temperature, a solution of 1.72 g of 1-cyanobenzotriazol (12 mmol) in 10 mL of anhydrous tetrahydrofuran is prepared. The solution thus obtained is brought to the temperature of 0° C., and at this temperature there is added, drop by drop and under vigorous stirring, 20 mL of a solution of [3-[i-(4-fluorophenyl)-1,3 dihydro-isobenzofiran-1-yl]propylldirnethylamine 5-magnesium bromide (6 nirnol) in tetrahydrofuran, prepared as in example 1, starting from 2.3 g of [3-[1-(4-fluorophenyl)-1,3 dihydro-isobenzofuran-1-yl]propyl]dimethylamine. Once the addition is completed, the solution is slowly brought to room temperature and kept under stirring overnight.

The reaction is extinguished, under vigorous stirring, by percolation of a solution of ammonium chloride. The solvent is then eliminated in a rotary evaporator, and the residue obtained is diluted with 100 mL of toluene. The organic solution is washed with 4 aliquots of 75 mL water. The organic extract is dehydrated with MgSO₄, and the solvent is eliminated by evaporation at reduced pressure, to obtain a dark-red oily residue. The crude residue obtained is purified via flash chromatography on 50 g of silica gel 70-230 mesh (eluent:toluene-isopropanol-triethylamine, 95-5-2, v/v), to obtain 1.39 g of pure product (molar yield 71.4%) having an NMR profile in accordance with the desired structure.

### EXAMPLE 5

Synthesis of Citalopram from Cyanogenous Chloride

In an inert atmosphere and with perfectly dehydrated apparatus at room temperature, a solution of 12.3 g of cyanogenous chloride (200 mmol) in 200 mL of anhydrous tetrahydrofuran is prepared. The solution thus obtained is brought to a temperature of −10° C., and at this temperature there is added, drop by drop and under vigorous stirring, 330 mL of a solution of [3-[1-(4-fluorophenyl)-1,3 dihydro-isobenzofuran-1-yl]propyl]dimethylamine 5-magnesium bromide (100 nmuol) in tetrahydrofuran, prepared as in example 1, starting from 37.8 g of [3-[1-(4fluorophenyl)-1,3 dihydro-isobenzofiran-1-yl]propyl]dimethylamine. Once the addition is completed, the solution is slowly brought back to room temperature and kept under stirring for one night. The reaction is then extinguished by percolation of the solution in a mixture of 150 mL of 30% ammonia and ice (300 g) under stirring. The mixture is subsequently brought to room temperature and then to a pH of approximately 5 with diluted hydrochloric acid, and extracted with 4 aliquots of 200 mL toluene.

The reunited organic extracts are washed with 200 mL of a saturated solution of sodium chloride. The solvent is eliminated by evaporation at reduced pressure, to obtain 40 g of oily residue with an HPLC titre of 72% with respect to the standard. After crystallization, 20.5 g of a product are obtained (molar yield 63.2%) with an HPLC titre of not less than 98% and with an NMR profile in accordance with the desired structure.

¹H NMR (200 MHz, CDCl₃): 7.60 (1H; s; 4-H), from 7.52 to 6.98 (6H; m; aromatic protons), 5.25 (1H; d; J=12.2; 3-CHₐ), 5.15 (1H; d; J=12.2; 3-CH_{b}), 3.08 (2H; t; J=7.5; 3′-CH₂), 2.71 (6H; s; —NCH₃), from 2.49 to 2.27 (2H; m; 1′-CH₂N) from 1.82 to 1.71 (2H; m; 2′CH₂). M/z ie 324 (M)⁺; 238 (M—CH₂CH₂CH₂(NCH₃)₂)⁺; 218 (m/z=238-HF)⁺.

1. A method for the manufacture of citalopram having the formula 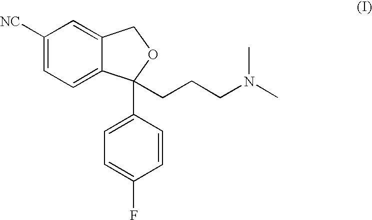
said method comprising the steps of:
(i) reacting 1-(4′-fluorophenyl)-1-(3-dimethylaminopropyl)-5-halophthalane having the formula 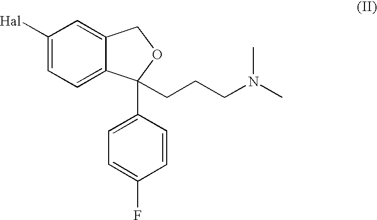
wherein Hal is halogen, with activated magnesium, to form the Grignard reagent of formula 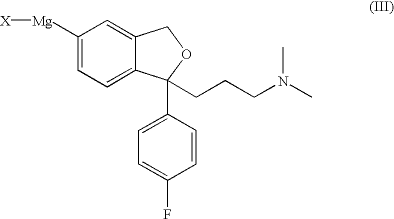
wherein X is halogen, followed by
(ii) reacting the compound of formula (III) with a compound containing a —CN group bound to a leaving group, to form citalopram.

2. The method of claim 1 wherein, in step (i), the compound of formula (II) is dissolved in an organic solvent (solution b) and added to a mixture of activated magnesium in an organic solvent (solution a).

3. The method of claim 2 wherein the compound (II) is used in a weight ratio with respect to magnesium of from 5:1 to 15:1.

4. The method of claim 3, wherein the weight ratio of compound (II) to magnesium is 7.5:1.

5. The method of claim 2, wherein the concentration of compound (II) in solution b is from 0.7 M to 1.2 M.

6. The method of claim 2, where the volume of solution a is from 40% to 60% with respect to the volume of solution b.

7. The method of claim 2 wherein the time within which solution b is added is more than 5 hours.

8. The method of claim 2 wherein the compound of formula (II) is used in a molar ratio with respect to magnesium of 1:2; the concentration of the compound of formula (II) in solution b is 1 M; the volume of solution a is 50% with respect to the volume of solution b; and the time within which solution b is added is from 6 to 8 hours.

9. The method of claim 1 wherein said compound containing a —CN group bound to a leaving group is selected from compounds having the formulas [chemical structure]
